(19) United States
(12) Patent Application Publication
(10) Pub. No.: US 2023/0138977 A1
(43) Pub. Date: May 4, 2023

(54) DEVICE AND SYSTEM FOR MONITORING OPERATION OF BODILY JOINTS

(71) Applicant: Goniotape, LLC, Reading, PA (US)

(72) Inventors: Sarah Rothenberger, Conshohocken, PA (US); Antonia Trupio, Farmingdale, NJ (US); Cecilia Roling, Odenton, MD (US); Samantha Burns, Delran, NJ (US)

(21) Appl. No.: 17/975,128

(22) Filed: Oct. 27, 2022

Related U.S. Application Data
(60) Provisional application No. 63/272,808, filed on Oct. 28, 2021.

(51) Int. Cl. A61B 5/11; A61B 5/00

(52) U.S. Cl. CPC A61B 5/1118; A61B 5/1123; A61B 5/6801; A61B 2562/0219

(57) ABSTRACT

A wearable monitoring device has an elongated adhesive-bearing body that includes a controller and at least one sensor operatively connected to the controller. The sensors are disposed and configured to capture data associated with bodily joint movement when worn overlying and/or adjacent the joint. The sensors may capture data relating to one or more of angle of flexion, repetitions, range of motion, speed, body temperature, moisture, pH level, atmospheric conditions, and location. Captured data may be interpreted to identify movement parameters and compare them to applicable movement limits, such as those associated with post-operative instructions. Movements exceeding limits may result in alerts/warnings to the wearer, via an associated smartphone or wearable notification device. Movement and/or alert data may be transmitted to the user's smartphone, etc., to a centralized monitoring system, and/or to a healthcare provider's and/or electronic medical records system, to facilitate recordkeeping and patient monitoring.

### CROSS-REFERENCE TO RELATED APPLICATION

This application claims the benefit of priority, under 35 U.S.C. § 119(e), to U.S. Provisional Patent Application No. 63/272,808, filed Oct. 28, 2021, the entire disclosure of which is hereby incorporated herein by reference.

### FIELD OF THE INVENTION

The present invention relates generally to biomedical devices and more particular to a wearable biomedical device and system usable for monitoring operation of bodily joints, e.g., in a pre- or post-surgical context.

### DISCUSSION OF RELATED ART

Injuries to and degeneration of the joints of the body often precipitate a need for joint replacement surgery or other medical interventions. By way of example, hip replacement surgery is a common procedure in geriatric and older adult populations. Knee, neck, elbow, shoulder and other orthopedic joint surgeries and replacements are also common. Approaches to surgery occur anteriorly, posteriorly, and laterally resulting in diverse types of precautions a patient needs to adhere to before and/or after surgery.

One of the roles of an occupational therapist is to teach precautions to patients after they have had surgery and to help patients safely complete activities throughout their day. Common methods for teaching precautions include hands-on training, visual handouts, and providing adaptive equipment training. However, while all these methods are used, it is believed that only 23% of patients follow precautions after surgery. As a result, patients are often readmitted to surgery, sometimes costing hospitals about $9,000 in readmission costs, and patients between $10,000 and

The problems behind these readmission costs include patients having differing levels of health literacy, minimal patient training time due to quick discharges, cognitive decline and memory deficits, and non-compliance with precautions and adaptive equipment.

What is needed is a device capable of monitoring operation of bodily joints, e.g., in a post-surgical context, to help promote the patient's adherence to post-surgical guidance to facilitate recuperation and/or avoid injury.

### SUMMARY

The present provides a device and system capable of monitoring operation of bodily joints, e.g., in a post-surgical context, to help promote the patient's adherence to the patient's post-surgical guidance to facilitate recuperation and/or avoid injury. A wearable monitoring device has an elongated adhesive-bearing body that includes a controller and at least one sensor operatively connected to the controller. The sensors are disposed and configured to capture data associated with bodily joint movement when worn overlying and/or adjacent the joint. The sensors may capture data relating to one or more of angle of flexion, repetitions, range of motion, speed, body temperature, moisture, pH level, atmospheric conditions, and location. Captured data may be interpreted to identify movement parameters and compare them to applicable movement limits, such as those associated with post-operative instructions. Movements exceeding limits may result in alerts/warnings to the wearer, via an associated smartphone or wearable notification device. Movement and/or alert data may be transmitted to the user's smartphone, etc., to a centralized monitoring system, and/or to a healthcare provider's and/or electronic medical records system, to facilitate recordkeeping and patient monitoring.

### BRIEF DESCRIPTION OF THE FIGURES

For a better understanding of the present invention, reference may be made to the accompanying drawings in which:

FIGS. 1A and 1B are front and right side views, respectively, of an exemplary tape-type wearable monitoring device for monitoring operation of bodily joints in accordance with an exemplary embodiment of the present invention;

FIGS. 2A-2D are right, left, front, and rear side views, respectively, of the wearable monitoring device of FIGS. 1A and 1B, shown mounted on a hip region of the body;

FIGS. 3A and 3B are front and rear views, respectively, of the wearable monitoring device of FIGS. 1A and 1B, shown mounted on a knee region of the body;

FIGS. 4A and 4B is a left side views of three wearable monitoring devices of FIGS. 1A and 1B, shown mounted on the hip and knee regions, respectively of the body;

FIGS. 5A-5D are right, left, front and rear side views, respectively, of the wearable monitoring device of FIGS. 1A and 1B, shown mounted on a torso region of the body;

FIGS. 6A-6D are right, left, front and rear side views, respectively, of the wearable monitoring device of FIGS. 1A and 1B, shown mounted on an elbow region of the body;

FIGS. 7A and 7B are top views of exemplary thin film pressure sensors of the prior art that used in the devices of FIGS. 1A-6D;

FIG. 8 is a top view of an exemplary temperature sensor of the prior art that used in the devices of FIGS. 1A-6D;

FIG. 9 is a system diagram showing an exemplary network computing environment in which the present invention may be employed;

FIG. 10 is a schematic block diagram of a Patient Computing Device in accordance with an exemplary embodiment of the present invention;

FIG. 11 is a schematic block diagram of a Biofeedback Device Monitoring System in accordance with an exemplary embodiment of the present invention; and

FIGS. 12A and 12B are left side views of the device of FIGS. 1A-1B, showing the device in use in unflexed and flexed leg states, respectively.

### DETAILED DESCRIPTION

The present invention provides a device and system capable of monitoring operation of the body, and particularly bodily joints, e.g., in a post-surgical context, to help promote the recuperating patient's adherence to their post-surgical guidance to facilitate recuperation and/or avoid injury. Accordingly, the present invention provides a wearable biofeedback device combining the practicality of goniometer measurements for medical professionals and the convenience of adhesive-lined elastic fabric tape for consumers and patients. More particularly, the device may be constructed of e-textile fabrics or the like, and is configured to be worn with one or more bend sensors spanning to a bodily joint, and to measure data including but not limited to the range of motion/angle, goniometry, rotation, velocity, repetitions of the limbs/joint of a patient's body.

Additionally, the device may be configured to include one or more other sensors relating to the joint or other bodily aspects that may be relevant to the joint of the body more broadly, and to capture/measure associated data including but not limited to moisture at the skin, pH at the skin, body temperature, and environmental aspects such as humidity, ambient temperature, atmospheric pressure.

Further, the device includes a controller that may incorporate hardware and/or software for interpreting data captured by the sensors and/or for communicating, e.g., wirelessly, the raw data or data associated with interpreted to a communications network or other computing device or system components, and allowing for information reporting to the system/system devices and the patient and/or the patient's caregivers, e.g., by transmitting corresponding data to a secure smartphone/tablet “app” or the like, and/or to the patient's electronic health record (EHR) as part of an electronic medical record (EMR) system.

In certain embodiments, the device may be paired with an alert bracelet, a smartphone/tablet/wearable device app, and an in-home location tracking system. In such embodiments, the device may work as an external goniometer, and can also track when a patient is moving a joint outside of a range of movement allowed by post-surgery precautions. For example, when a patient is about to extend a joint past a permitted range, the app will detect this non-recommended operation of the joint using data collected via the device, and alert the patient on a wearable bracelet or other-type notification device before a post-surgery precaution is broken. Alternatively, these functions may be performed by a controller of the device.

In certain embodiment, the device may be configured to include one or more location sensors, such as a GPS-based device of a type commercially available from Apple Computer of Cupertino, Calif. as a Tile or an AirTag. Somewhat similarly, the device may communicate with “tiles” or location beacons, and can also track when a patient is completing movements outside of a range allowed by post-surgery precautions. For example, when a patient is moving in a way that is non-compliant with post-operative or other activity guidance/instructions, then the app will detect this non-recommended activity using data collected via the device, and alert the patient on a wearable bracelet or other-type notification device, For example, the notification device may be configured to alert the patient through auditory, visual, or tactile information. This works well because immediate feedback is provided to the patient when they are breaking precautions. Additionally, the tiles are used to gather data as to where in the home, etc. the non-compliant movements were made. This allows healthcare providers, therapists and/or caregivers to address specific activities, transfers or adaptive techniques with the patient in an individualized manner.

By way of further example, the device could be utilized both preoperatively and postoperatively. Preoperative use allows patients to learn their precautions prior to surgery in a more effective way and anticipate needed environmental adaptations within their homes.

Still further, the device could be used in the athletics/sports industry, to continuously measure joint range of motion of its wearer, and to send corresponding information to an app for analysis and tracking, and to have sent to the wearer corresponding notifications via bracelet/notification device. For example, the device/system could be used to assist athletes and coaches in measuring performance and in acting as a preventative precaution to avoid injury. By way of further example, the device may also be used in the contexts of hand therapy and neurorehabilitation (with emphasis on neuromuscular rehabilitation in this setting) to track range of motion (e.g. in degrees) after injury, surgery, or onset of neurological condition (e.g. stroke), and send corresponding information to a care team. For example, the device/system could be used to assist therapists and doctors in measuring improvements in range of motion.

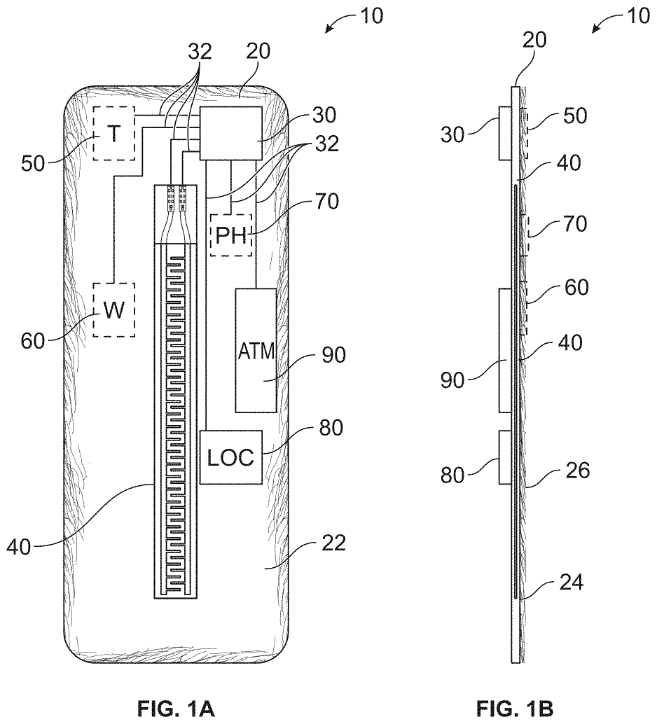

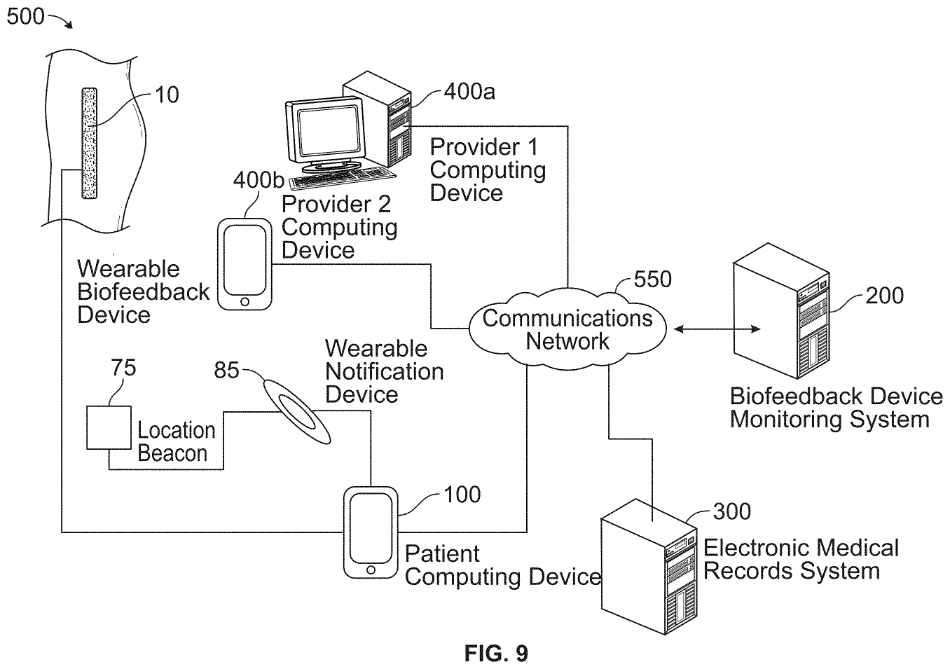

According to illustrative embodiment(s) of the present invention, various views are illustrated in FIGS. 1A-13 and like reference numerals are used consistently throughout to refer to like and corresponding parts of the invention for all of the various views and figures of the drawings.

The following detailed description of the invention contains many specifics for the purpose of illustration. Any one of ordinary skill in the art will appreciate that many variations and alterations to the following details are within scope of the invention. Accordingly, the following implementations of the invention are set forth without any loss of generality to, and without imposing limitations upon, the claimed invention.

Wearable Monitoring Device

FIGS. 1A and 1B are front and right side views, respectively, of an exemplary tape-type wearable device 10 for monitoring operation of bodily joints in accordance with an exemplary embodiment of the present invention. By way of example, the wearable device may be worn to monitor activity of the hip, knee, elbow joints as well as activity of the spine. In this exemplary embodiment, the wearable monitoring device 10 has the form of an adhesive-lined fabric tape, and may be manufactured to have a form factor and a tape body somewhat similar to conventional kinesiology tape. However, in accordance with the present invention, the device 10 includes hardware sensors and a controller comprising hardware and/or software components that may be used to store raw data, interpret raw data, and/or transmit raw and/or interpreted data to another computing device.

Referring now to FIGS. 1A and 1B, it will be appreciated that this exemplary monitoring device 10 includes a tape body 20 having a top side 22 and a bottom side 24. The bottom side 24 is provided with adhesive 26 that may be used to adhere the device 10 directly to the skin of a person, e.g., overlying or adjacent a limb joint. The tape body 20 may be constructed of any suitable woven or non-woven material, as known in the art.

The device 10 further includes a number of hardware sensors 40, 50, 60, 70, 80, 90 (adapted to generate data) and a controller 30 (adapted to acquire/receive the sensor data and store, process/interpret and/or transmit data) supported on the tape body 20. The sensors and controller 30 may be supported on the tape body 20 in any suitable manner. In certain embodiments, the sensor may be disposed within the thickness of the tape body 20, which in other embodiments the sensor may be supported on, or be exposed to, the outer surface 22 or inner surface 24 of the tape body 20. For example, if it is desirable that a particular sensor be in contact with the skin, that sensor may be mounted on the inner surface 24 of the tape body 20. By way of further example, if it is desirable that a particular sensor be exposed to/open to the ambient environment, that sensor may be mounted on the outer surface 22 of the tape body 20. In the exemplary embodiment shown in FIGS. 1A-1B, the controller 30, environmental sensor 90 and location sensor 80 are shown as disposed on the outer surface 22 of the tape body 20, the bend sensor 40 is shown as disposed within a thickness of the tape body 20, and the temperature sensor, moisture sensor 60, pH sensor 70 are shown as disposed on the inner surface 24 of the tape body 20, so as to abut the user's skin, or closely overlie the skin, e.g., if the tape body 20 is ultimately adhered to a garment. It should be noted that in FIGS. 1A and 1B, the placement of the components relative to each other, the thickness of the tape band, arrangement of the adhesive, etc. are shown exaggerated for exemplary and illustrative purposes only. Any suitable arrangement of the sensors on or within the tape body may be used.

The sensors 40, 50, 60, 70, 80, 90 are operatively connected to the controller 30. In the exemplary embodiment, the sensors and controller are operatively connected by conductors 32. The conductors, sensor and/or controller may be part of an integral body in the nature of a flexible or other printed circuit board, or may be discrete components interconnected by discrete conductors. In certain embodiments, the tape body 20 may be constructed of a woven or fabric material in which the conductors are provided as conductive (e.g., silver or conductive material-coated) strands and/or other conductors disposed within the weave or fabric material.

The controller 30 may include a battery (e.g., a conventional lithium ion or similar battery, a fuel cell, etc.), and communication hardware (e.g., a Bluetooth communication hardware) to facilitate wireless data communication with the sensors and/or an external computing device such as a Patient Computing Device (PCD) 100 running a companion “app.” Further, the controller may include a processor, a memory and instructions stored in the memory and executable by the processor for receiving data from the sensors, storing the data, processing/interpreting the data (e.g., via software, a conditioning circuit or the like), and/or transmitting data (raw data or interpreted data) to a computing device.

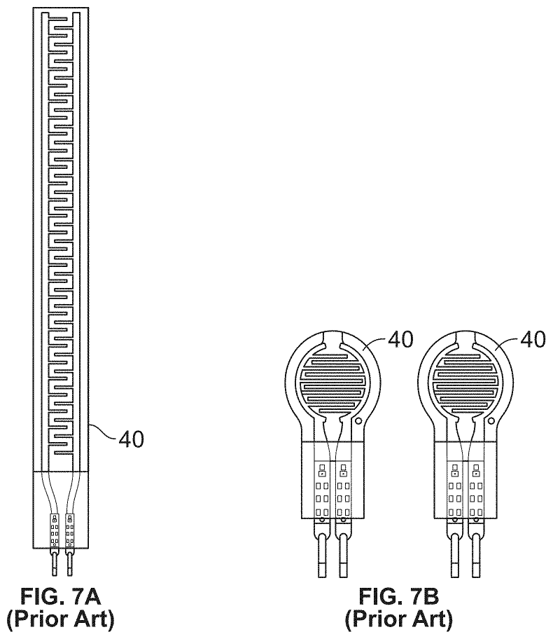

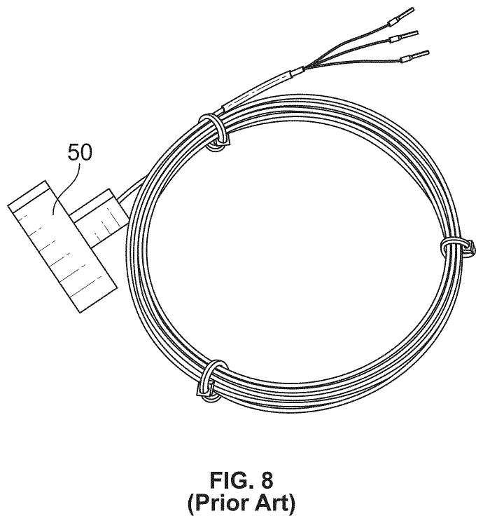

Referring again to FIGS. 1A and 1B, the exemplary device 10 includes a bending sensor 40, which may be any conventional sensor capable of generating data as a function of bending of, e.g., out of the plane of the sensor. Any suitable sensor may be used for this purpose. By way of example, a flexible thin film, force sensitive resistor-type sensor may be used for this purposes. As known in the art, such a sensor can detect bending and/or pressure on the sensor and provide an output signal/data (a measure of resistance across the sensor) that varies according to the degree of bending/pressure of the sensor. Exemplary prior art bending sensors 40 of this type are shown by way of example in FIGS. 7A and 7B.

The sensors are provided for measuring a range of motion (ROM) (e.g., in degrees), angle of joint rotation, velocity or movement, number of flexion repetitions, acceleration, resistance, and/or distance of an associated joint, limb or other associated body portion during movement. Alternatively, or additionally, other sensors including but not limited to a gyroscope, accelerometer, pedometer, goniometer, odometer/speedometer or velocity sensor may be employed for these purposes. Any desired sensor may be used to gather data while the patient is in motion, gathering information in real time during activity.

Associated data gathered from the sensors may be received by the controller 30, where it may be processed and/or interpreted for users to identify range of motion (ROM) (e.g., in degrees), angle of joint rotation, velocity or movement, number of flexion repetitions, acceleration, resistance, and/or distance of an associated joint, limb or other associated body portion during movement, etc. Additionally, the interpreted data can then be used to identify precaution breaks, risk for injury and potential re-injury, methods for movement optimization, and areas for therapeutic intervention, etc. In other embodiments, the sensors or controller may merely transmit raw data to another device, such as the PCD 100 or BDMS 200, for such processing, interpretation, etc.

Additionally, other sensors may be included for measuring other patient and/or environmental aspects, such as a body temperature sensor 50, user moisture (sweat) sensor 60, pH sensor 70, atmospheric humidity, temperature, pressure sensor 90, etc. Suitable sensors may be employed for this purpose. Associated data gathered from the sensors may be received by the controller 30, where it may be processed and/or interpreted to provide relevant information. FIG. 8 is a top view of an exemplary temperature sensor 50 of the prior art that used in the monitoring device 10.

Additionally, a location sensor 80 may be included for identifying a location of the device, e.g., using geospatial coordinates, proximity sensors, etc. For example, GPS hardware, or Tile, AirTag or a location beacon-sensor hardware may be employed for this purpose. Associated data gathered from the sensor 80 may be received by the controller 30, where it may be processed and/or interpreted to provide relevant information.

Accordingly, the sensors of the device generate data during joint/limb/body movement, and may transmit the associated data to the PCD 100 or BDMS 300 for interpretation/analysis (or may alternatively interpret/analyze such data at the controller 30). The data and associated movements are analyzed to determine whether the movements are compliant or non-compliant with post-surgical or other guidelines, to provide insight into the nature, quality and safety of movements during activity, and/or for other purposes, accordingly to suitable logic. When non-compliance (e.g., outside of pre- or post-surgical guidelines) or other monitored conditions are found, the device and/or system may trigger other events, such as providing sensory feedback to the patient via a notification device, or by sending data to another system component.

System Environment

An exemplary embodiment of the present invention is discussed below for illustrative purposes. FIG. 1 is a system diagram showing an exemplary network computing environment 500 in which the present invention may be employed. In accordance with the present invention, the network 500 includes a wearable monitoring device 10.

Additionally, as shown in FIG. 1, the exemplary network environment 500 includes conventional computing hardware and software for communicating via a communications network 550, such as the Internet, etc., using Healthcare Provider Computing Devices 400a, 400b and a Patient Computing Device 100, which may be, for example, one or more personal computers/PCs, laptop computers, tablet computers, smartphones, or other computing devices.

In accordance with a certain aspect of the present invention, the network computing environment 500 may also include conventional computing hardware and software as part of a conventional electronic medical records system, such as an EPIC, Cerner or ALLSCRIPTS system, which are referred to collectively herein as an Electronic Medical Records (EMR) System 300, and may receive patient-related data to be stored in the patient's corresponding electronic health record (EHR). The EMR System 300 may interface with the Patient and/or Provider Computing Devices 100, 400a, 400b and/or other devices as known in the art. These systems may be existing or otherwise generally conventional systems including conventional software and web server or other hardware and software for communicating via the communications network 550. Consistent with the present invention, these systems may be configured, in conventional fashion, to communicate/transfer data via the communications network 550 with the Biofeedback Device Monitoring System (BDMS) 200 in accordance with and for the purposes of the present invention, as discussed in greater detail below.

Accordingly, the network computing environment 10 includes the monitoring device 10, and the BDMS 200, which is operatively connected to one or more of the monitoring device, PCD 100, Provider Computing Devices 400a, 400b and/or the EMR System 300, for data communication via the communications network 550.

For example, the monitoring device 10 may communicate with the Patient Computing Device 100 and/or BDMS 200 may gather patient-related data from the Patient Computing Device 100 via the communications network 550. Further, for example, the BDMS 200 may process received data and/or transmit data to the Provider Computing Devices 400a, 400b and/or to the EMR System 300, via the communications network 550. Accordingly, the BDMS 200 may function as a communication portal between patients and providers, data logs, connections to the electronic health record, and instructional guides on precautions, and can act as an information center. The app may have an accessibility center that allows for adjusting font size, contrast, read aloud, speech to text, etc.

Hardware and software for enabling communication of data by such devices via such communications networks are well known in the art and beyond the scope of the present invention, and thus are not discussed in detail herein.

In this exemplary embodiment of the present invention, the network 500 further includes a notification device. The notification device may be the PCD 100 or a separate wearable notification device 85, such as a wearable notification bracelet. In the exemplary embodiment shown, a biofeedback device comprises at least two discrete components, namely, the wearable monitoring device 10 that serves as a monitoring device for receiving motion-related inputs from operation of the joint/limbs/body of the wearer, and a wearable notification device 85, for delivering notifications to the wearer under certain conditions. In one exemplary embodiment, the wearable monitoring 20 has the form of an adhesive-lined fabric tape, and the wearable notification device 85 has the form of a wearable bracelet. In other embodiments, these devices may have different forms and/or be integrated into a single physical device.

For example, the wearable notification device 85 may include hardware and/or software similar to that of wearable activity trackers, smart watches and the like, and thus may include, for example, lights/LEDs for providing a visual alert, audio speakers or other hardware for providing an audible alert, and/or a vibration motor or the like for providing a haptic/vibratory alert, as well as a suitable battery and suitable communication hardware (such as Bluetooth hardware), e.g., for data communication with the PCD 100 and/or its app. Accordingly, for example, non-compliance or other monitored events is determined, the device or app/PCD 100 may transmit appropriate data to the wearable notification device 85 to cause it to provide a suitable alert (illumination, sound or vibration) to alert the wearer.

In certain embodiments, the network environment 500 further includes location tiles or location beacons 75. In such embodiments, such location beacons 75 may be placed in commonly used spaces of a client's home, workplace, etc. that are relevant to the patient's condition. Such location beacons may be placed throughout the home/workplace, etc., so that the patient's current location can be logged and correlated with patient activity, detected by the device 10, that triggers an alert, warning. For example, it may be found that post-surgical precautions for total hip replacement (as determined by the monitoring device 10 and/or PCD 100) are often violated when the patient is in the bathroom (e.g., as determined by location sensor 80 or by proximity to a location beacon 75 placed in the patient's bathroom), which would create an opportunity for a caregiver to provide the patient with an instruction/recommendation to avoid sitting on a low toilet, or to use a device, such as a commode, etc.

The wearable monitoring device 10, wearable notification device 85 and/or PCD 100 may be configured to communicate with such location beacons 75 in a manner known in the art. The notification device 85 may thereby gather data as to which room/location the patient is in during non-compliant movement, and may then transmit that data to the PCD 100, device 10, Provider Computing Device 400a, 400b and/or the BDMS 200.

Patient Computing Device

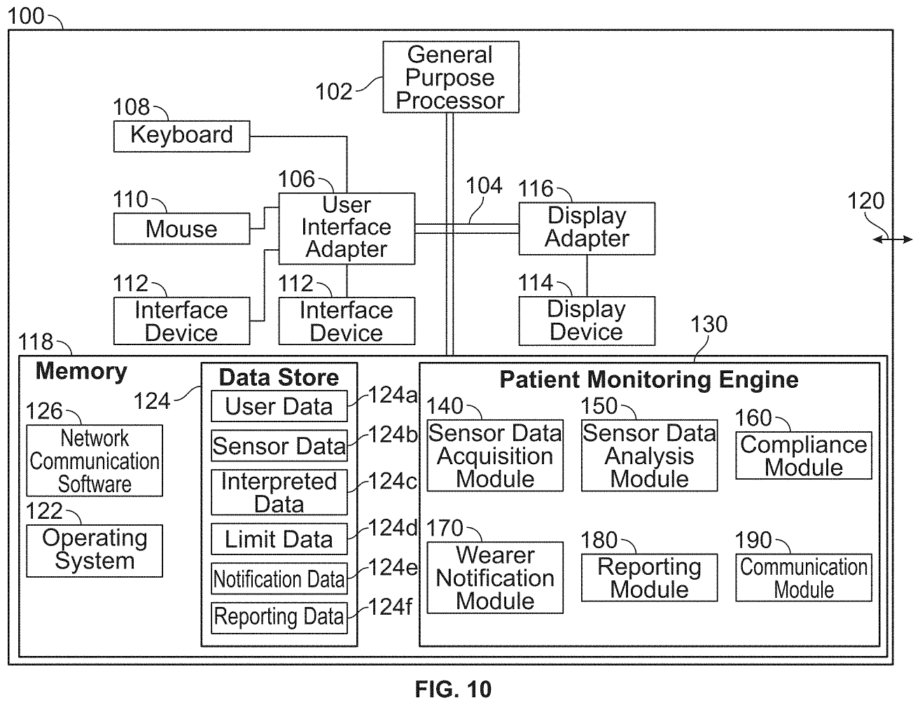

FIG. 10 is a schematic block diagram showing an exemplary Patient Computing Device (PCD) 100 in accordance with an exemplary embodiment of the present invention. The exemplary PCD 100 is a special-purpose computer system that includes conventional computing hardware storing and executing both conventional software enabling operation of a general-purpose computing system, such as operating system software, network communications software, and specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

Referring again to FIG. 10, there is illustrated a block diagram of an exemplary PCD 100 according to some embodiments is shown. In some embodiments, the PCD 100 may, for example, execute, process, facilitate, and/or otherwise be associated with the embodiments and methods described herein.

Accordingly, the exemplary PCD 100 of FIG. 10 includes a general-purpose processor, such as a microprocessor (CPU), 102 and a bus 104 employed to connect and enable communication between the processor 102 and the components of the presentation system in accordance with known techniques. According to some embodiments, the processor 102 may be or include any type, quantity, and/or configuration of processor that is or becomes known. In some embodiments, the processor 102 may comprise multiple inter-connected processors, microprocessors, and/or micro-engines. According to some embodiments, the processor 102 (and/or the system 100 and/or other components thereof) may be supplied power via a power supply (not shown), such as a battery, an Alternating Current (AC) source, a Direct Current (DC) source, an AC/DC adapter, solar cells, and/or an inertial generator. In the case that the system 100 comprises a server, such as a blade server, necessary power may be supplied via a standard AC outlet, power strip, surge protector, and/or Uninterruptible Power Supply (UPS) system.

The exemplary PCD 100 includes a user interface adapter 106, which connects the processor 102 via the bus 104 to one or more interface devices, such as a keyboard 108, mouse 110, and/or other interface devices 112, which can be any user interface device, such as a touch-sensitive screen, digitized entry pad, etc. The bus 104 also connects a display device 114, such as an LCD screen or monitor, to the processor 102 via a display adapter 116.

The bus 104 also connects the processor 102 to memory 118, which can include a hard drive, a solid-state drive, an optical drive, a diskette drive, a tape drive, etc. The memory 118 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 118 may, according to some embodiments, store one or more software components. Any or all of the exemplary instructions and data types described herein and other practicable types of data may be stored in any number, type, and/or configuration of memory systems that is or becomes known. The memory 118 may, for example, comprise one or more data tables or files, databases, table spaces, registers, and/or other storage structures. In some embodiments, multiple databases and/or storage structures (and/or multiple memory systems) may be utilized to store information associated with the system 100. According to some embodiments, the memory 118 may be incorporated into and/or otherwise coupled to the system 100 (e.g., as shown) or may simply be accessible to the system 100 (e.g., externally located and/or situated).

The PCD 100 may communicate with other computers or networks of computers, for example via a communications channel, network card, modem or transceiver (collectively, “transceiver”) 120. In some embodiments, the transceiver 120 may comprise any type or configuration of communication system that is or becomes known or practicable. The transceiver 120 may, for example, comprise a Network Interface Card (NIC), a telephonic system, a cellular network system, a router, a hub, a modem, and/or a communications port or cable. According to some embodiments, the transceiver 120 may also or alternatively be coupled to the processor 102. In some embodiments, the transceiver 120 may comprise an IR, RF, Bluetooth™, Near-Field Communication (NFC), and/or Wi-Fi® network system coupled to facilitate communications between the processor 102 and another system (not shown). The JACD 200 may be associated with such other computers in a local area network (LAN) or a wide area network (WAN), and may operate as a server in a client/server arrangement with another computer, etc. Such configurations, as well as the appropriate communications hardware and software, are known in the art.

In some embodiments, the PCD 100 may not be specially configured in accordance with the present invention. Rather it may be merely conventional hardware and software, and may be used in accordance with the present invention to navigate and/or interact with web pages delivered by the BDMS 200 for the purposes described herein.

In the embodiment shown in FIG. 10, the PCD 100 is specially configured in accordance with the present invention. Accordingly, as shown in FIG. 10, the PCD 100 includes computer-readable, processor-executable instructions stored in the memory 118 for carrying out the methods described herein. Further, the memory 118 stores certain data, e.g., in one or more databases or other data stores 124 shown logically in FIG. 10 for illustrative purposes, without regard to any particular embodiment in one or more hardware or software components.

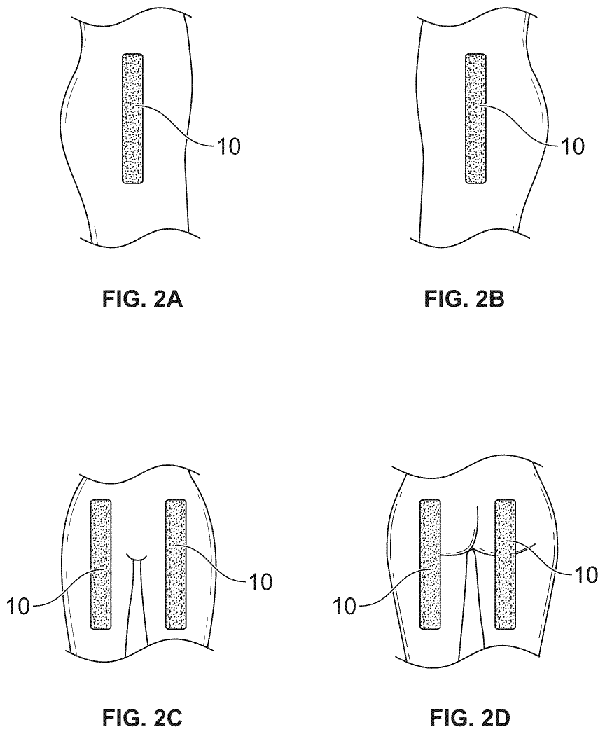

Further, as will be noted from FIG. 10, the PCD 100 includes, in accordance with the present invention, a Patient Monitoring Engine (PME) 130, shown schematically as stored in the memory 118, which includes a number of additional modules providing functionality in accordance with the present invention, as discussed in greater detail below. These modules may be implemented primarily by specially-configured software including microprocessor—executable instructions stored in the memory 118 of the PCD 100. Optionally, other software may be stored in the memory 118 and and/or other data may be stored in the data store 124 or memory 118. Further, the UIME 130 includes one or more modules shown logically in FIG. 2 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

As shown in FIG. 10, the PCD 100 includes a data store 124 and a PME 130 in accordance with the present invention. The PME is operable to receive sensor-obtained data and/or data derived from the sensor-obtained data that may be used to monitoring the patient's bodily movements and/or associated bodily and/or environmental conditions, as discussed in greater detail below.

In part, the PCD 100 stores User Data 124a in the data store 124, e.g., in a database cluster. The User Data 124a identifies the user and includes any relevant user profile data, such as user identification information, patient/insured identification information, monitoring device identification information and/or healthcare provider contact information. By way of example, some or all of this information may be provided by or gathered from the user by direct input via the PCD 100 or by data communication via the network 550 with a Provider Computing Device 400a, 400b, an EMR System 300 and/or a BDMS 200.

Further, the exemplary PCD 100 stores Sensor Data 124b in the data store 124. The Sensor Data 124b is data gathered from sensors e.g., 40, 50, 60, 70, 80, 90 of the monitoring device 10. Accordingly, this data may be raw data gathered by such sensors and then transmitted to the PCD 100 by the controller 30 of the monitoring device 10, to permit the CPD 100 to process/interpret such raw data. For example, the Sensor Data 124b may include raw resistance value data captured by the bend sensor 40.

In other embodiments, raw sensor data may not be received by the PCD 100. Rather, the raw sensor data may be processed/interpreted by the controller 30 of the monitoring device 10, and the PCD 100 may receive, e.g., instead of sensor data, data resulting from the processing/interpreting of the sensor data at the monitoring device 10, and may store such interpreted data as Interpreted Data 124c. For example, the Interpreted Data 124c, may include angle of flexion, flexion repetition data, range of motion data, speed data, etc.

The PME 130 includes a Data Acquisition Module (DAM) 140 that is operable to receive raw data received from sensors of the monitoring device 10, and to store such data in the data store 124 of the memory 118 as Sensor Data 124b. For example, this may include receiving raw sensor data transmitted from the controller 30 and/or sensors of the monitoring device 10. For example, raw sensor data from the bend sensor 40 may include a series of resistance values over time, as a result of varying resistance during flexion of a joint resulting in bending of the bend sensor 40 of the monitoring device 10 worn over the joint.

In this example, in which raw sensor data is received by the PCD 100 and processed by the PCD 100, the PME 130 further includes a Sensor Data Analysis Module (SDAM) 150 that is operable to process or otherwise interpret the raw Sensor Data 124b to produce Interpreted Data 124c, which may be stored in the data store 124 in the memory 118. provide measurements, calculations or other determinations, as desired, as a function of the raw sensor data. For example, the Interpreted Data 124c corresponding to the raw Sensor Data 124b from the bend sensor 40 may be a range of motion expressed in degrees, an angle of flexion expressed in degrees, a repetition count expressed as a whole number, etc. The processing/interpretation may be performed according to stored logic of the SDAM 150, and may involve any suitable algorithms, signal processing, comparisons, calculations, etc. to produce any desired information, such as a range of motion, angle of flexion, repetition count, etc.

The PME 130 further includes a Compliance Module (CM) 160 that is operable to determine whether the wearer's activities, as captured by the sensors of the monitoring device 10, are compliant with any applicable limits for user activity, e.g., as may be assigned by a healthcare provider as post-surgical restrictions during post-surgical recovery. For example, a limit may require the patient to limit movement of a particular joint to 30 degrees of flexion during a certain post-surgical timeframe. This may be performed by the CM 160 comparing Limit Data 124d, stored in the data stored 124 of the memory 118, to current activity data as reflected in the Interpreted Data 124c.

The PME 130 further includes a Wearer Notification Module (WNM) 170 that is operable to provide a suitable notification to a wearer of the monitoring device 10, when the CM 160 determines that the wearer's activities, as captured by the sensors of the monitoring device 10, are not compliant with applicable limits for user activity. In certain embodiments, this involves issuing a suitable alert signal via the PCD 100, e.g., as a vibration, audible tone, or textual or graphical message produced or displayed via hardware of the PCD 100. In other embodiments, this involves communicating (e.g., acting in concert with the COM module 190) with an external device, such as a wearable notification device 85, so that the wearable notification device 85 issues a suitable alert signal, e.g., as a vibration, audible tone, or textual or graphical message produced or displayed via hardware of the wearable notification device 85. This may be performed by the WNM 170 referencing stored Notification Data 124e, stored in the data stored 124 of the memory 118, to identify a suitable manner for notifying the user, and obtaining relevant data for doing so. In certain embodiments, the nature and/or content of the alert signal may be customizable and/or individualized.

The Notification Data may be predetermined/prestored/hard-coded, e.g., as part of the app, and stored in the memory 118 of the PCD 100. Alternatively, customized Notification Data may be provided by the user via the PCD 100, a healthcare provider via a Provider Computing Device 400a, 400b and/or via the BDMS 200, on a per-patient basis, and then may be stored as appliable Notification Data 124e In the latter case, the WNM 170 may be responsible for receiving and storing the Notification Data 124e.

The PME 130 further includes a Reporting Module (RM) 180 that is operable to provide suitable information to at least one of the EMR System 300 and the healthcare provider, via the Provider Computing Device 400a, 400b. The RM 180 may provide reporting according to any suitable logic or algorithm of the RM 180. For example, the RM 180 may be configured to report information when the CM 160 determines that the wearer's activities, as captured by the sensors of the monitoring device 10, are not compliant with applicable limits for user activity. Alternatively, for example, the RM 180 may simply report activity data at a predetermined time or periodically, e.g., weekly, without regard to compliance or non-compliance. This involves communicating (e.g., acting in concert with the COM module 190) with an external device, such as the EMR System 300 and/or the Provider Computing Device 400a, 400b. This may be performed by the RM 180 referencing stored Reporting Data 124f, stored in the data stored 124 of the memory 118, to identify a suitable manner for reporting the relevant information for the user, and obtaining relevant data for doing so.

The Reporting Data may be predetermined/prestored/hard-coded, e.g., as part of the app, and stored in the memory 118 of the PCD 100. Alternatively, customized Reporting Data may be provided by the user via the PCD 100, a healthcare provider via a Provider Computing Device 400a, 400b and/or via the BDMS 200, on a per-patient basis, and then may be stored as appliable Reporting Data 1244 In the latter case, the RM 190 may be responsible for receiving and storing the Reporting Data 124f.

The PME 130 further includes a Communications Module (COM) 190 that is operable to work in tandem with other modules of the PME 130 to communicate with external computing devices in support of the functionality described above.

Accordingly, when prescribed precautions (e.g., maximum degree of flexion) are broken (in other words, are not complied with), the location sensor and/or beacons provide information indicating where the patient was when the non-compliance occurred. This information may be reported to clinicians, and be used by the clinicians to customize occupational therapy and physical therapy sessions to focus on the areas of the home in which non-compliance tends to occur, as well as identify adaptive devices, durable medical equipment, or adaptive techniques to implement for the patient, thus improving safety and reducing risk of re-injury.

Biofeedback Device Monitoring System

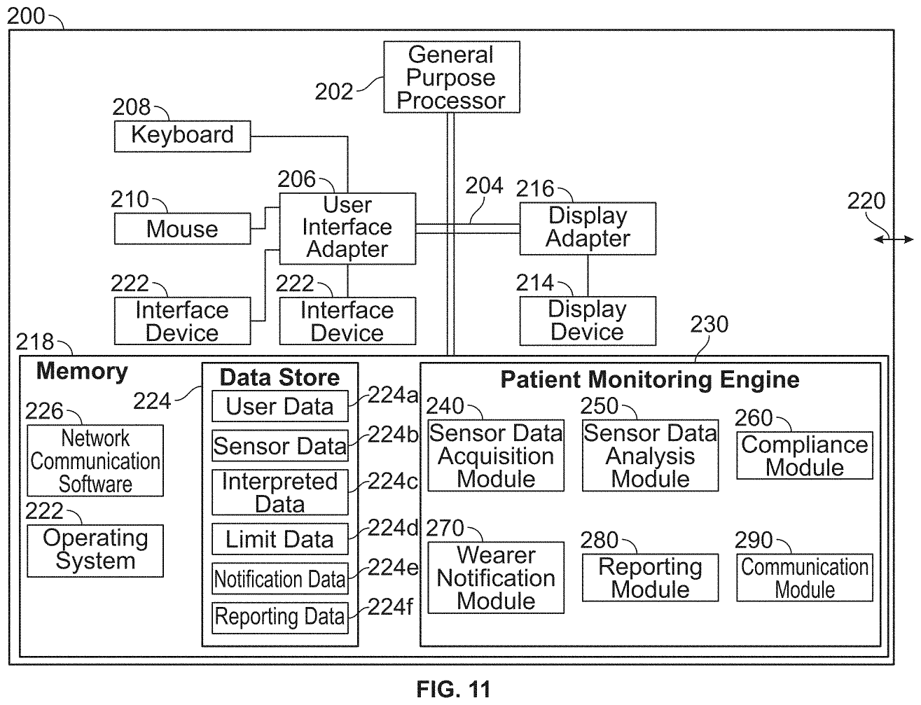

FIG. 11 is a schematic block diagram showing an exemplary Biofeedback Device Monitoring System (BDMS) 200 in accordance with an exemplary embodiment of the present invention. The exemplary BDMS 200 is a special-purpose computer system that includes conventional computing hardware storing and executing both conventional software enabling operation of a general-purpose computing system, such as operating system software, network communications software, and specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

The BDMS 200 may include any or all of the hardware, software, data, Patient Monitoring Engine and Modules described above with reference to the PCD 100 of FIG. 10, Accordingly, the description above is incorporated herein by reference, but is not repeated here.

In certain embodiments, the monitoring device and/or any companion app of the PCD 100 is configured only to gather data and provide the patient/wearer with very basic information (e.g., what applicable precautions are, that there is non-compliance when it occurs, and an indication of where the patient/person was when the non-compliance occurred). The BDMS 200 may be configured to generate a more-detailed report (e.g., including data log, trends, etc.) for the clinician, and to provide/display more information than the app. Additionally, the BDMS 200 may provide for applicant precautions/parameters to be set by the clinician through the BDMS 200, in a manner such that patients are unable to after them.

System Operation

In use, the wearable monitoring device 10 may be applied as desired overlying and/or adjacent to any limbs, joints or bodily portions desired to be monitored. It should be noted that it may be desirable to orient a particular wearable monitoring device 10 relative to the body according to the particular structure and/or arrangement of sensors on the particularly wearable monitoring device. This may involve removing a release sheet to expose adhesive 26 on a bottom side 24 of the monitoring device 10, and pressing the device against the skin, tightly-fitting clothing, etc. This may involve disposing the monitoring device 10 in specific locations relative to the joint according to the construction of the monitoring device 10 and/or the nature and/or orientation of its sensors. In some embodiments, the adhesive and/or monitoring device may be configured to be readily removable and re-adherable, making it a readily re-usable device. In other embodiments, the adhesive and/or monitoring device may not be configured to be readily re-usable, and may instead be intended to be disposable after a single use, or for a single period, or until the adhesive or other components fail.

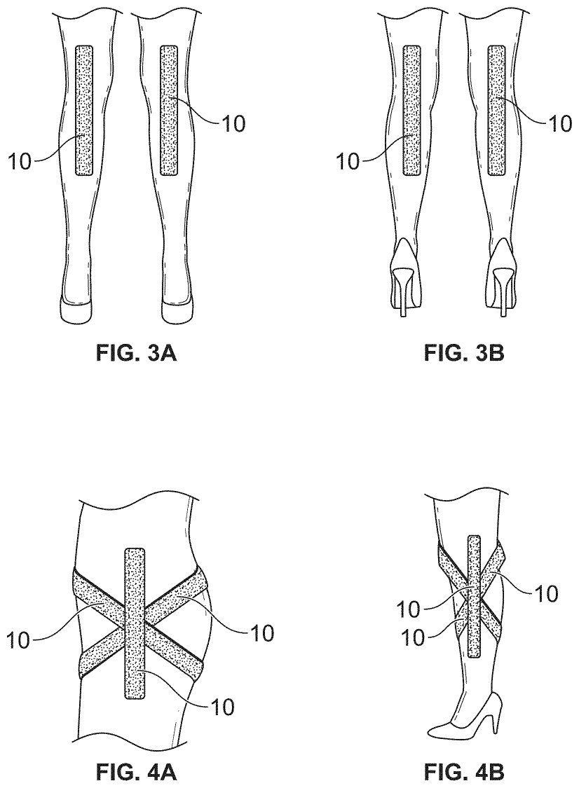

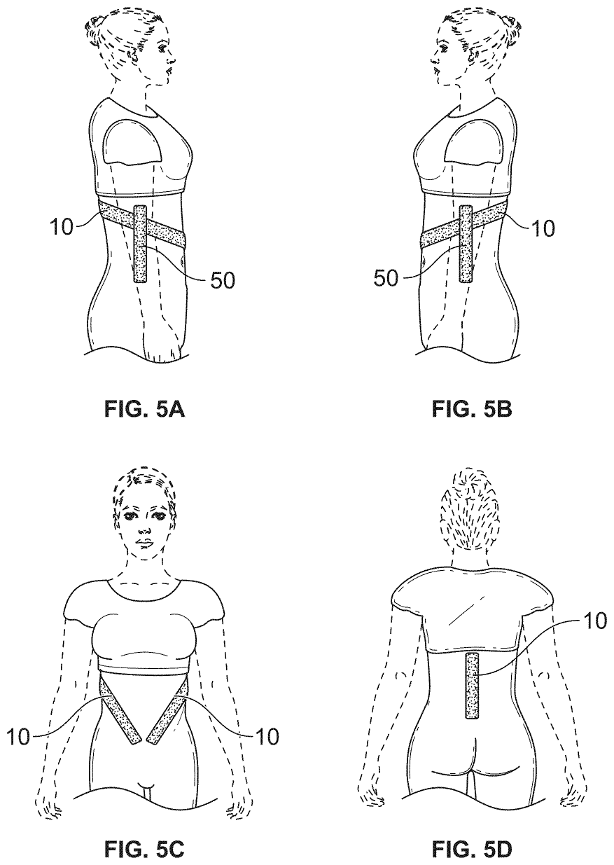

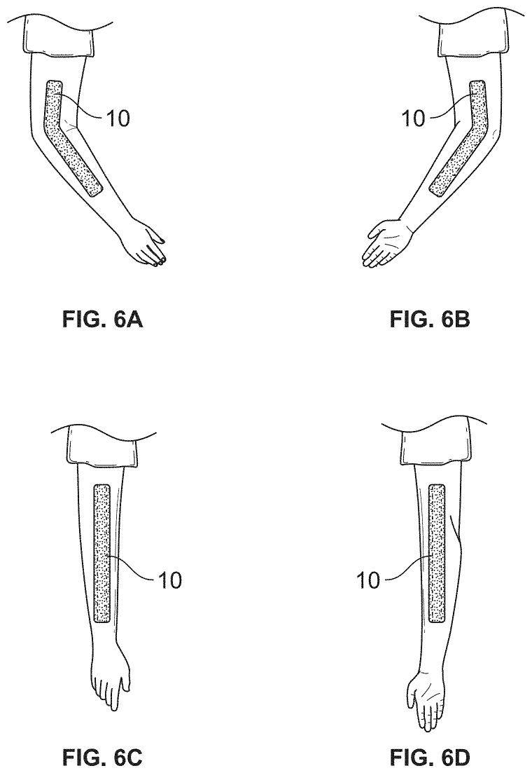

By way of example, FIGS. 2A-2D are right, left, front, and rear side views, respectively, of the wearable monitoring device 10 of FIGS. 1A and 1B, shown mounted in exemplary positions on a hip region of the body. FIGS. 3A and 3B are front and rear views, respectively, of the wearable monitoring device 20 shown mounted in exemplary positions on a knee region of the body. FIGS. 4A and 4B is a left side views of three wearable monitoring devices of FIGS. 1A and 1B, shown mounted in exemplary positions on the hip and knee regions, respectively of the body. FIGS. 4A and 4B illustrate that it may be desirable in certain embodiments to apply more than one device 10 adjacent a single joint to be monitored. In such embodiments, more than one device 10 may operate in tandem, and the data gathered from such devices 10 may be used together to interpret data and identify range of motion, angle of limb, degree of flexion, repetitions, speed, etc. FIGS. 5A-5D are right, left, front and rear side views, respectively, of the wearable monitoring device 10, shown mounted in exemplary positions on a torso region of the body. FIGS. 6A-6D are right, left, front and rear side views, respectively, of the wearable monitoring device 10, shown mounted in exemplary positions on an elbow region of the body.

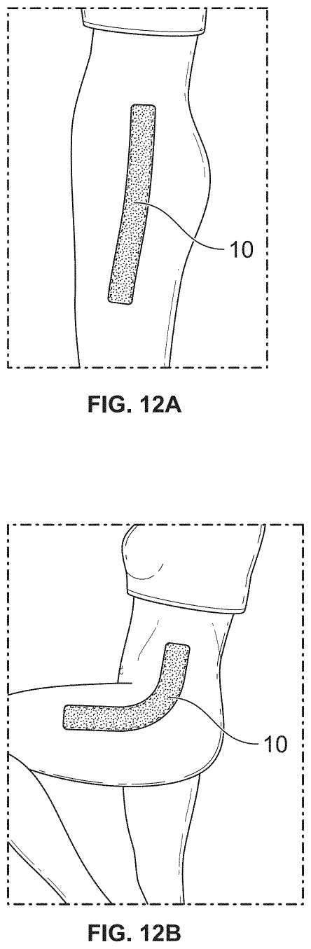

The user/wearer of the monitoring device 10 may then perform various activities, including daily living activities and/or therapeutic activities/interventions. Such activities may involve flexion of a joint being monitored by the monitoring device 10. By way of example, FIGS. 12A and 12B are left side views of the device of FIGS. 1A-1B, showing the device in use with the hip in unflexed (FIG. 12A) and flexed (12B) states, respectively. As a result of the flexion, for example, the bend sensor 40 may be correspondingly bent, as will be appreciated from FIGS. 12A and 12B. This results in generation/creation of raw sensor data by the bend sensor 40 of the monitoring device. Similarly, the other sensors of the monitoring device will generate/create corresponding sensor data.

Raw data from the sensors is captured by the controller 30 of the monitoring device 10 operatively connected to the various sensors. The controller 30 may subsequently communicate the raw sensor data to another device, such as the PCD 100 or BDMS 300, which may process/interpret the raw sensor data. In other embodiments, the controller 30 may process/interpret the sensor data, and therefore the controller 30 may include modules, store data, etc. that is described herein in relation to the PCD 100 and BDMS 300.

The PME 130 (or PME 230 of the BDMS 200) then processes or otherwise interprets the raw Sensor Data to produce Interpreted Data providing measurements, calculations or other determinations, as desired, as a function of the raw sensor data. For example, the Interpreted Data corresponding to the raw Sensor Data from the bend sensor 40 may be a range of motion expressed in degrees, an angle of flexion expressed in degrees, a repetition count expressed as a whole number, etc. The processing/interpretation may be performed according to stored logic of the SDAM 150, and may involve any suitable algorithms, signal processing, comparisons, calculations, etc. to produce any desired information, such as a range of motion, angle of flexion, repetition count, etc. This information may be displayed at the PCD 100, e.g., under control of a Display Module of the PME 130 (and/or the PME 230 of the BDMS 200). Alternatively, or additionally, this information may be communicated to the DMBS 200, Provider Computing Device 400a, 400b and/or stored in the EMR System 300, e.g., for later reference and review, and to support a healthcare provider in caring for a patient.

The PME 130 (or PME 230 of the BDMS 200) then determines whether the wearer's activities, as captured by the sensors of the monitoring device 10, are compliant with any applicable limits for user activity, e.g., by comparing Limit Data to current activity data as reflected in the Interpreted Data 124c. Accordingly, for example, it may be determined whether limits assigned by a healthcare provider as post-surgical restrictions during post-surgical recovery are being adhere to. Notably, this may be performed in real time, e.g., concurrently with the user's performance of the activity generating the raw sensor data, so that appropriate feedback may be given to the person during performance of the activity.

Standardized or generic Limit Data 124d reflecting appropriate limits for the user may be predetermined/prestored/hard-coded, e.g., as part of the app, and stored in the memory 118 of the PCD 100. Alternatively, customized Limit Data (or a customized selection of standardized limit data) may be provided by ay healthcare provider via a Provider Computing Device 400a, 400b and/or via the BDMS 200, on a per-patient basis, and then may be communicated to the PCD 100 for storage as applicable Limit Data 124d. In certain instances, the limit data may include location data. In the latter case, the CM 160 may be responsible for receiving and storing the Limit Data 124d as well as performing the comparison referenced above.

In certain embodiments, when the PME 130 (or PME 230 of the BDMS 200) determines that the wearer's activities, as captured by the sensors of the monitoring device 10, are not compliant with applicable limits for user activity, the PME 130, etc. may issue a notification via a Wearer Notification Module (WNM) 170, which is operable to provide a suitable notification to a wearer of the monitoring device 10. The notification alerts may be issued via the PCD 100 (e.g., displayed via the PCD 100) or via a separate wearable notification device 85. The notification alerts provided by this system may be particularly useful helping patients to understand the application of precautions to activities of daily living and/or instrumental activities of daily living, and by providing reminders, particular for patients with cognitive decline and/or with memory deficits.

In certain embodiments, the network environment 500 further includes location tiles or location beacons 75. In such embodiments, such location beacons 75 may be placed in commonly used spaces of a client's home, workplace, etc. that are relevant to the patient's condition. Such location beacons may be placed throughout the home/workplace, etc., so that the patient's current location can be logged and correlated with patient activity, detected by the device 10, that triggers an alert, warning. For example, it may be found that post-surgical precautions for shoulder surgery (as determined by the monitoring device 10 and/or PCD 100 are most often violated when the patient is in the kitchen (as determined by sensor 80 or by proximity to a location beacon 75 in the patient's kitchen), which would create an opportunity for a caregiver to provide the patient with an instruction/recommendation to avoid reaching for items on upper kitchen cabinet shelves, or to move those items to lower shelves, etc.

The wearable monitoring device 10, wearable notification device 85 and/or PCD 100 may be configured to communicate with such location beacons 75 in a manner known in the art. The notification device 85 may thereby gather data as to which room/location the patient is in during non-compliant movement, and may then transmit that data to the PCD 100, device 10, Provider Computing Device 400a, 400b and/or the BDMS 200.

Accordingly, the system (e.g., at the BDMS 200 and/or at the PCD 100) may thereby maintain a running log of all precaution (limit) breaks and near-breaks, as well as motion analysis reports. In certain embodiments, the PME 130 (or PME 230 of the BDMS 200) provides suitable information to at least one of the EMR System 300 and the healthcare provider, via the Provider Computing Device 400a, 400b. Accordingly, the system may provide for transmission of associated data to a Provider Computing Device 400a, 400b, e.g., to alert a caregiver, and/or to the BMDS 200 for maintaining a detailed log of data to track patient progress over time and analyze effectiveness of therapeutic intervention and/or to the patient's EHR record in the EMS 300.

The monitoring device 10 and/or PCD 100 may include many accessibility features including customizable visual color contrast, customizable font size and style, read-aloud options, the ability to zoom in/out, ability for the patient to control via verbal commands, configurable button/keyboard size and reactivity, and image descriptions.

Accordingly, the present invention provides or supports a financial benefit for hospitals, decreased readmission rates, decreased correctional surgeries, quicker/earlier discharge, financial, physical, and emotional benefits for patients, increased safety and precaution adherence, decreased stress regarding safety and discharge, decreased risk of correctional surgery, financial benefits for outpatient clinics, and increased services pre- and post-operatively. The present invention thereby increases patient adherence to surgical precautions during everyday activities to improve patient safety, function, and independence, and to increase financial benefits for healthcare providers across settings.

Further still, the present invention provides for immediate feedback to the client with respect to bodily movements and potentially harmful activity, healthcare provider/therapist tracking via EMR, etc., alerts to prevent a motion that breaks movement precaution guidelines, increases patient safety and confidence while decreasing readmission to hospitals, and is able to quantify/measure adherence to post-surgical precautions. Accordingly, the present invention has application to various patient populations, and offers particular benefits in the context of orthopedic, neurologic and cardiothoracic surgeries.

The various implementations and examples shown above illustrate an exemplary method and system. As is evident from the foregoing description, certain aspects of the present implementation are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present implementation. Accordingly, the specification and drawings are to be regarded in an illustrative rather than a restrictive sense.

Certain systems, apparatus, applications or processes are described herein as including a number of modules. A module may be a unit of distinct functionality that may be presented in software, hardware, or combinations thereof. When the functionality of a module is performed in any part through software, the module includes a computer-readable medium. The modules may be regarded as being communicatively coupled. The inventive subject matter may be represented in a variety of different implementations of which there are many possible permutations.

The methods described herein do not have to be executed in the order described, or in any particular order. Moreover, various activities described with respect to the methods identified herein can be executed in serial or parallel fashion. In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

In an exemplary embodiment, the monitoring device is a machine that operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a smart phone, a web appliance, a network router, switch or bridge, or any machine capable of executing a set of instructions (sequential or otherwise) that specify actions to be taken by that machine or computing device. Further, while only a single machine is illustrated, the term “machine” shall also be taken to include any collection of machines that individually or jointly execute a set (or multiple sets) of instructions to perform any one or more of the methodologies discussed herein.

The example computer system and client computers include a processor (e.g., a central processing unit (CPU) a graphics processing unit (GPU) or both), a main memory and a static memory, which communicate with each other via a bus. The computer system may further include a video/graphical display unit (e.g., a liquid crystal display (LCD) or a cathode ray tube (CRT)). The computer system and client computing devices also include an alphanumeric input device (e.g., a keyboard or touch-screen), a cursor control device (e.g., a mouse or gestures on a touch-screen), a drive unit, a signal generation device (e.g., a speaker and microphone) and a network interface device.

The system may include a computer-readable medium on which is stored one or more sets of instructions (e.g., software) embodying any one or more of the methodologies or systems described herein. The software may also reside, completely or at least partially, within the main memory and/or within the processor during execution thereof by the computer system, the main memory and the processor also constituting computer-readable media. The software may further be transmitted or received over a network via the network interface device.

The term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing or encoding a set of instructions for execution by the machine and that cause the machine to perform any one or more of the methodologies of the present implementation. The term “computer-readable medium” shall accordingly be taken to include, but not be limited to, solid-state memories, and optical media, and magnetic media.

It should be appreciated that the exemplary embodiment described above is for illustrative purposes only, and non-limiting. While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A wearable monitoring device comprising:
an elongated body having an adhesive disposed on a side thereof;
at least one sensor operatively supported on said elongated body, said at least one sensor being operable to gather sensor data associated with at least one of a range of motion, an angle of flexion, an angle of rotation, a velocity, a number of repetitions, an acceleration, a resistance, and a distance of movement of an associated body portion when said elongated body is mounted by said adhesive adjacent said associated body portion during movement of said body portion; and
a controller supported on said elongated body and operably connected to said at least one sensor to receive data from said at least one sensor during movement of said body portion.

2. The wearable monitoring device of claim 1, wherein said elongated body comprises a woven fabric.

3. The wearable monitoring device of claim 2, wherein at least one of said at least one sensor and a conductor operatively connecting said at least one sensor to said controller is woven into said fabric.

4. The wearable monitoring device of claim 1, wherein said at least one sensor comprises a bend sensor capable of generating sensor data as a function of bending of the bend sensor.

5. The wearable monitoring device of claim 4, wherein said bend sensor comprises a force sensitive resistor.

6. The wearable monitoring device of claim 1, further comprising at least one of a temperature sensor, a moisture sensor, a pH sensor, a location sensor, and an environmental humidity sensor, an environmental temperature sensor, and an environmental pressure sensor.

7. The wearable monitoring device of claim 1, wherein said at least one sensor comprises at least one of a gyroscope, an accelerometer, a pedometer, a goniometer, an odometer, a speedometer, and a velocity sensor.

8. The wearable monitoring device of claim 1, wherein said controller is configured to interpret sensor data to determine at least one of the range of motion, the angle of flexion, the angle of rotation, the velocity, the number of repetitions, the acceleration, the resistance, and the distance of movement of the associated body portion.

9. The wearable monitoring device of claim 8, wherein said controller is further configured to:
determine whether sensor data received from said at least one sensor indicates non-compliance with at least one predetermined activity guideline; and
if it is determined that said sensor data received from said at least one sensor indicates non-compliance, then initiate an alert notification to a wearer of the wearable monitoring device.

10. The wearable monitoring device of claim 8, wherein said controller is configured to initiate said alert notification via at least one of an LED configured to provide a visual alert, audio hardware configured to provide an audible alert, and a vibration motor to provide a vibratory alert.

11. The wearable monitoring device of claim 8, wherein said controller is configured to initiate said alert notification to the wearer of the wearable monitoring device by causing the wearable monitoring device to deliver said alert notification.

12. The wearable monitoring device of claim 8, wherein said controller is configured to initiate said alert notification to the wearer of the wearable monitoring device by transmitting data to a portable computing device to cause the portable computing device to deliver said alert notification.

13. The wearable monitoring device of claim 8, wherein said controller is configured to initiate said alert notification to the wearer of the wearable monitoring device by transmitting data to a wearable notification device to cause the wearable notification device to deliver said alert notification.

14. The wearable monitoring device of claim 1, wherein said controller comprises data communication hardware configured to transmit data to a computing device configured to interpret sensor data to determine at least one of the range of motion, the angle of flexion, the angle of rotation, the velocity, the number of repetitions, the acceleration, the resistance, and the distance of movement of the associated body portion.

15. The wearable monitoring device of claim 14, wherein said computing device is configured to:
determine whether sensor data received from said at least one sensor indicates non-compliance with at least one predetermined activity guideline; and
if it is determined that said sensor data received from said at least one sensor indicates non-compliance, then initiate an alert notification to a wearer of the wearable monitoring device.

16. The wearable monitoring device of claim 9, further comprising a location sensor, said controller being configured to capture location data associated with non-compliance when said controller determines that sensor data indicates non-compliance.

17. A wearable monitoring device comprising:
an elongated body having an adhesive disposed on a side thereof;
a bend sensor operatively supported on said elongated body, said at least one sensor being operable to gather sensor data as a function of bending of said bend sensor as a result of movement of a bodily joint when said elongated body is mounted by said adhesive adjacent said associated body portion during movement of said bodily joint; and
a controller supported on said elongated body and operably connected to said at least one sensor to receive data from said at least one sensor during movement of said body portion.

18. The wearable monitoring device of claim 17, wherein said bend sensor comprises a force sensitive resistor.

19. The wearable monitoring device of claim 17, wherein said controller is configured to interpret sensor data to determine at least one a range of motion, an angle of flexion, an angle of rotation, a velocity, a number of repetitions, an acceleration, a resistance, and a distance of movement of an associated bodily joint when said elongated body is mounted by said adhesive adjacent said bodily joint during movement of said bodily joint.

20. The wearable monitoring device of claim 17, wherein said controller is further configured to:
determine whether sensor data received from said at least one sensor indicates non-compliance with at least one predetermined activity guideline; and
if it is determined that said sensor data received from said at least one sensor indicates non-compliance, then initiate an alert notification to a wearer of the wearable monitoring device.

21. The wearable monitoring device of claim 9, further comprising a location sensor, said controller being configured to capture location data associated with non-compliance when said controller determines that sensor data indicates non-compliance.

22. A biofeedback monitoring system comprising:
a wearable monitoring device comprising:
an elongated body having an adhesive disposed on a side thereof;
at least one sensor operatively supported on said elongated body, said at least one sensor being operable to gather sensor data associated with at least one of a range of motion, an angle of flexion, an angle of rotation, a velocity, a number of repetitions, an acceleration, a resistance, and a distance of movement of an associated body portion when said elongated body is mounted by said adhesive adjacent said associated body portion during movement of said body portion; and
a controller supported on said elongated body and operably connected to said at least one sensor to receive sensor data from said at least one sensor during movement of said body portion, said controller being configured to transmit controller data; and
a computing device comprising:
a processor; and
a memory operatively connected to said processor, said memory storing executable instructions that, when executed by the processor, causes the computing device to:
analyze controller data received from said controller of said wearable monitoring device;
determine whether said controller data received indicates non-compliance with at least one predetermined activity guideline; and
if it is determined that said controller data indicates non-compliance, then initiate an alert notification to a wearer of the wearable monitoring device.

23. The system of claim 22, wherein said controller data comprises said sensor data.

24. The system of claim 22, wherein said controller data comprises interpreted data, said controller producing said interpreted data as a function of said sensor data.

25. The system of claim 22, further comprising:
a notification device comprising:
data communication hardware; and
at least one of an LED operable to provide a visual alert, audio hardware operable to provide an audible alert, and a vibration motor operable to provide a vibratory alert, in response to receipt of a signal for initiating an alert from said computing device.
